(19) United States
(12) Patent Application Publication
(10) Pub. No.: US 2009/0112239 A1
(43) Pub. Date: Apr. 30, 2009

(54) Sticky dilatation balloon and methods of using

(75) Inventor: John Thao To, Newark, CA (US)

Correspondence Address: Katherine N. Addison Esq., 1930 Whitley Avenue, Apt 104, Hollywood, CA, 90068, US

(73) Assignee: SPECIALIZED VASCULAR TECHNOLOGIES, INC., Fremont, CA (US)

(21) Appl. No.: 12/262,409

(22) Filed: Oct. 31, 2008

Related U.S. Application Data
(60) Provisional application No. 61/001,217, filed on Oct. 31, 2007.
(60) Provisional application No. 61/002,343, filed on Nov. 8, 2007.

(51) Int. Cl. A61B 17/221; A61B 17/22

(52) U.S. Cl. 606/159; 606/194; 604/103.05; 606/200

(57) ABSTRACT

The present invention involves an expandable element with an outer sticky surface. The expandable element may be in the form of a balloon with deflated and inflated configurations. The expandable element serves to both dilate a lumen in a blood vessel thereby opening it and to exert force upon the sticky surface in order to press it into apposition against a vessel wall. The outer sticky surface may be provided directly upon the expandable element or on a separate outer sheath that conforms to and follows the contours of the expandable element. The sticky surface may take the form of a biochemical composition and/or a mechanically abrasive structure such as microhooks, hairs, mesh netting, etc. Optionally, additional expandable elements may be provided proximal and distal to the main element to occlude blood flow on either side of a lesion in order to better trap emboli for collection.

### BACKGROUND OF THE INVENTION

1. Field of the Invention

The present invention relates to the field of medical devices for preventing embolization characterized by the obstruction of blood flow in a vessel or organ by an embolus or undissolved foreign particle (i.e. thrombi, tissue fragments, clumps of bacteria, protozoan parasites, fat globules, or gas bubbles). More specifically, the invention relates to non-prosthetic, temporary treatment devices for reducing the risk of embolization at any stage of a stenting procedure. Most specifically, the invention relates to proactive, expandable devices with debris-grabbing adhesive surfaces and their methods of use.

2. Description of the Related Art

Presently, embolic protection devices are used for procedures that entail a high risk of embolization with adverse consequences. These procedures include carotid artery stenting (CAS), renal artery stenting (RAS), and vein graft stenting (VGS). General approaches include distal balloon occlusion (Percusurg GuardWire) with aspiration, filter devices (Angioguard, Filterwire, EmboShield, Spider), and flow reversal devices (ArteriA).

The distal occlusion device and the filter device suffer from having to place a bulky device far distal to the target lesion. This position can cause complications such as spasm and can allow embolization out a branch just distal to the lesion and proximal to the embolic protection device (i.e. especially a problem with RAS). These conventional devices are also bulky and stiff and can cause embolization in crossing the lesion in the first place. The filter devices have pore sizes that must be big enough (i.e. at least around 100 microns) not to get clogged up. The compromise is that as a result many smaller particles pass through. At the end of the procedure the filter devices have retrieval failures and the balloon occlusion devices have aspiration inadequacy that can result in complications and embolization. The flow reversal device is bulky (10 French OD) and interrupts flow as does the distal occlusion device. All of these devices require many extra steps complicating the procedure and extend the procedure time significantly.

Another shortcoming of traditional embolic protection devices is that they only protect from embolization acutely. Open space between stent struts allows for particles to loosen from the lesion and embolize post procedure. This occurs usually around 3 days post-procedure in CAS and is a significant subacute embolization problem that is currently not addressed.

Some devices have attempted to reduce the risk of embolization by cleaning and/or dilating the area of the lesion before, during, and after placement of a stent across it. The present invention improves upon these methods with a simpler, safer, and more effective design.

United States Patent Application Publication No. (hereinafter US Pub. App.) 20070213753 entitled “Stent-cleaning assembly and method” by David F. Waller (Tampa, Fla.) and assigned to Wilson-Cook Medical Inc. (Winston-Salem, N.C.) discloses a device for cleaning a stent with an elongate catheter body, a shaft therein, an engagement member to hold the device inside the stent, a handle, and a cleaning member. The publication focuses selectively on cleaning an occluded stent as an alternative to balloon compression of plaque, co-luminal insertion of a smaller stent, and stent replacement ([0005]-[0008]). The present invention also covers cleaning the vessel itself with or without the stent, including before a stent is introduced.

A shortcoming of US Pub. App. '753 is that is fails to provide devices and methods for the safe insertion of the stent-cleaning device. The present invention includes dilation elements and filters that extend distally and proximally to a lesioned and/or stented region in order to temporarily control blood flow and eliminate the risk of embolization during vessel and/or stent cleaning and dilatation. Another shortcoming of the publication is that it requires separate elements for the cleaning member and the engagement means. A brush with bristles is recited for the cleaning member (i.e. see claims 6-13 and 19) while several elements, including hooks, are recited for the engagement means ([0020]). In the present invention the same element, including hooks, can be used to provide both a debris removal (cleaning) and lumen gripping (engagement) function.

Although the publication discloses a balloon with an adhesive or abrasive surface generally ([0040]) it does not teach several more detailed aspects of the present invention including: using aligned fibers to form the adhesive surface, a detachable adhesive coating transferable from the balloon to the luminal wall, and an adhesive coating layer with a thickness that is less than a height of the debris trapping structure(s) embedded therein. US Pub. App. '753 also does not enable an adhesive surface formed of any particular chemical or biological composition and there is no mention of any of the material compositions of the present invention (3,4-L-dihydroxyphenylalanine, poly(dopamine methacrylamide-co-methoxyethylacrylate), dihydroxyphenyl, hydroxyphenylalanine, albumin, collagen, fibrin, fibrinogen, cyanoacrylate, acrylated urethane, polyurethane, silicone, elastomeric rubber, epoxy, a water-activated adhesive, and a pressure-activated adhesive).

United States Patent No. (hereinafter USP) U.S. Pat. No. 6,626,861 entitled “Balloon catheter apparatus and method” by Charles C. Hart, et al. and assigned to Applied Medical Resources (Santa Margarita, Calif.) discloses a device for cleaning and removing material from within a vascular conduit or other bodily passageway. The device involves two coaxial tubular shafts within the catheter tube with a passageway for fluid defined between them. The device also involves a balloon with an annular balloon chamber in connection with the fluid passageway. On the outer surface of the balloon is an expandable mesh sleeve that is expandable and collapsible independently of the balloon.

One drawback of the device and methods disclosed in USP '681 is that teaches driving the mesh into the vascular conduit and abrading the surface of the vascular conduit with the sleeve (i.e. see Abstract, claim 32, 3:3-5, etc.). Since the mesh is all at one topographic level this may cause damage, tearing, and inflammation to healthy tissue in addition to the removal of debris. The present invention provides a better solution with a sticky adhesive surface or raised mechanically abrasive elements. The sticky adhesive surface can be gently pressed into debris without disturbing the smooth luminal walls of healthy vessels. The mechanical abrasive elements (i.e. hair, hooks, etc.) that are raised beyond the general surface of the balloon reach out to remove debris without the need drive the balloon into tissue. Another drawback is that since the balloon and its outer mesh sleeve are independently controllable, it permits the possibility of the balloon becoming kinked, tangled, displaced, or lost within the sleeve and/or between the mesh. In the present invention the adhesive or abrasive surface used to trap debris may exist directly upon the surface of the balloon itself. Alternatively, in another embodiment of the present invention, the sticky surface can be provided on a separate expandable cover sheath outside of the balloon in which the balloon and the cover sheath are attached and expandable/collapsible as a unit. USP '681 also fails to disclose embodiments other than mesh for forming an abrasive outer surface on the balloon's sleeve, including hooks, bristles, and hairs. Although woven fibers are disclosed (9:30), aligned fibers are not disclosed. Rather, USP '681 teaches that the plurality of filaments that form the sleeve may “be sized, shaped and oriented to adjust the irregularity or abrasiveness of the outer surface.” (11:13-15) This suggests an irregular rather than regular/aligned fiber/filament orientation. USP '681 focuses exclusively on a woven mesh to provide the vascular conduit cleaning function and does not teach adhesive chemical compositions or sticky coatings to bond to debris. The present invention includes both adhesive chemical compositions and mechanical abrasive debris-trapping means, independently or integrated in the same device, for synergistic superior conduit cleaning. Using a sticky coating alone or in addition to mechanically abrasive elements permits the removal of smaller particulates of plaque dust from a lesion site.

U.S. Pat. No. 5,904,698 entitled “Surgical shaving device for use within body conduits” by Thomas P. Thomas, et al. and assigned to Applied Medical Resources Corporation (Laguna Hills, Calif.), like USP '681, also discloses a device that relies upon a mesh network on an expandable outer sleeve to trap debris. The device of USP '698 is a shaving device in that it uses the mesh network similar to a razor blade or cheese grater to induce some debris to protrude through the mesh outer sleeve into the catheter tube where it is contact by a treatment element. The treatment element can be one or more electrode or a blade.

A drawback of this device is that it is not suited well for trapping finer particles of debris. There is no disclosure of using a sticky surface or chemical adhesive compositions together with the mesh. The present invention includes both sticky surfaces and abrasive surfaces to remove a greater variety of forms and sizes of debris. In addition, the mesh network assumes that the unhealthy tissue and debris to be shaved extends beyond the surface of the healthy tissue to be spared. However, in some cases, debris could be in a crevice, corner, or side branch. Unhealthy tissue with lesions could be embedded within the healthy tissue of the vasculature requiring it to be dug out. The mesh network is not suited for digging or reaching into a crevice. In some embodiments of the present invention mechanically abrasive elements that project from the surfaced of the balloon may be used to reach into crevices to grasp diseased tissue. The distal ends of the projecting elements may be coated with sticky chemical adhesives to aid this process and to collect even tiny particles of debris within crevices.

Another potential drawback of the invention of USP '698 is that the treatment element (i.e. electrode or blade) is within the catheter tube or on its surface (the mesh serving as one electrode) and is not capable of reaching out of the catheter tube. The sticky balloon of the present invention may itself have an extendable treatment element and is also adaptable for use with other surgical tools (i.e. cauterization and ablation probes) for treating tissue that can be easily manipulated and extended.

### BRIEF SUMMARY OF THE INVENTION

An alternative method for protecting against embolization in a stenting procedure is to use a dilatation balloon (or “Sticky Balloon”) with an outer surface that adheres to loosened tissue particles and other anatomic debris. The balloon's outer surface can grab and trap particles and debris by using either or both of a biochemical adhesive composition bonded to it or a covering comprised of a mechanically abrasive trapping structure (i.e. microhooks that function like Velcro™).

In a procedure, before stenting, the dilatable Sticky Balloon is collapsed in a smooth restraining sheath and advanced across the lesion. The restraining sheath is then pulled back to expose the balloon. The balloon is inflated to dilate the lesion. Dilatation causes pressure within the balloon to force the sticky outer surface directly into debris as it proceeds into apposition against the inner wall of a vessel. Tissue debris or emboli are trapped on the surface of the balloon. Loosened (i.e. pedunculated) tissue particles that are still connected to the inner vessel walls will also attach to the sticky surface. When the balloon is subsequently moved or deflated the adhesive or grasping strength holding particles to the surface is strong enough to detach their corporeal connections from the roots. Collecting loose particles from the vessel walls in this manner prevents the later inadvertent severance of particles which could lead to embolization when a severed particle becomes lost free floating and then lodged. After deflating the balloon it is pulled back into an expandable tube connected to the collection sheath.

Additionally, the Sticky Balloon can also be used within a stent after the stent has already been implanted in order to further expand the stent and trap emboli as it dilates. Sometimes the stent placement procedure irritates tissue on the sides of vessel walls to loosen or release emboli precursors. Using the sticky dilatation balloon immediately after placing the stent functions as a clean-up procedure to prevent acute embolization before it has a chance to occur. Typically, it is helpful, if not necessary, to further expand and dilate a stent anyways after implantation. Thus, this approach does not add any extra step but simply adds additional features to already existing steps: adhesion and particle attraction and retention.

Although the balloon itself provides dilatation, as an alternative to the sticky surface existing directly on the balloon the adhesion or material-grabbing function can also be provided by an expandable sheath over the dilatation balloon instead of it being on the balloon itself. For example, a thick biochemical adhesive composition and/or a complicated surface geometry/topography pattern for mechanical abrasion may be disposed on an outer sheath covering the balloon walls. This separation of the dilation and adhesion functions permits a greater degree of specialization and refinement without compromise of the materials chosen for the balloon walls and outer sheath. For example, to provide better dilation and safely withstand greater pressures within (without the risk of a bursting rupture), the balloon walls may be formed of a thicker material without worrying about the flexibility of the material (for better tissue-grabbing maneuverability) or the ability of an adhesive coating to be integrated therein. Rather, these criteria for material selection (i.e. flexibility, ability to bond to an adhesive coating, etc.) are made a priority for the outer sheath material.

Another advantage of delegating the function of tissue-grabbing to the sheath when using a mechanically abrasive structure is that it avoids making the fluid-filled balloon wall more susceptible to rupture. Etching or patterning methods used to produce a tissue-grabbing surface geometry (including the creation of microhooks, indentations, mesh, etc.) may at the same time weaken the strength of the surface material. By providing the tissue-grabbing surface on an outer sheath the strength of the balloon walls is not compromised while the sheath (potentially with an intricate tissue-grabbing design) remains protected from high pressures by the balloon walls.

In a preferred embodiment of the present invention the sticky dilatation balloon is dumbbell-shaped to trap the debris better. An extension of this concept is for the catheter upon which the main lesion-traversing balloon is mounted to incorporate additional balloons for deployment distal and proximal to the lesion. These outer balloons are preferably occlusion balloons for obstructing blood flow around the lesion to prevent embolization while the center sticky dilatation balloon is placed. The outer occlusion balloons also expedite and facilitate the trapping of debris to the surface of the center sticky balloon by preventing debris from leaving the area of the lesion.

In an alternative embodiment of the present invention the sticky surface of the balloon (or of the optional sheath conforming to its outer surface) deposits a thin layer of a protective coating onto the inner lumen of a narrowed region of the vessel (i.e. over the lesion). The coating sticks to the lumen surface and holds any unstable plaque in place to avoid its interference with hemodynamics from uneven peripheral contours. Preferably, the coating is composed of aligned nanofibers to encourage rapid endothelization. (See co-pending commonly owned non-provisional patent application Ser. No. 12/128,533 entitled “Coatings for promoting endothelization of medical devices” for exemplary coatings.)

The sticky dilatation balloon of the present invention may be obtained as an individual component for use with pre-existing standard catheter systems. Preferably, the balloon is acquired with a complementary catheter as part of an angioplasty balloon catheter system. By simply inflating the balloon against the plaque, the system binds to the unstable plaque and collects it for removal through the catheter lumen. The balloon can be made flexible and with a sufficiently small profile in its deflated condition to be able to cross a tight lesion without disrupting plaque during the initial insertion. An additional embolic protection mechanism does not have to be deployed outside the lesion. Unstable plaque is removed directly from the vessel wall while free floating plaque is rounded up and condensed so that no potential emboli are left behind to cause subacute embolization. These preventative and proactive features of the present invention coupled with its ease of use (at any time before, during, or after stenting) make it particularly advantageous and an improvement over the reference art.

### BRIEF DESCRIPTION OF THE SEVERAL VIEWS OF THE DRAWINGS

FIG. 1 shows a dilatation balloon in an expanded configuration, mounted uniformly along and around an elongated catheter body, having a sticky outer surface comprising a chemical adhesive.

FIG. 2 shows a dilatation balloon in an expanded configuration, mounted uniformly along and around an elongated catheter body, having an outer surface capable of trapping debris with structurally abrasive surface elements such as microhooks.

FIG. 3 shows a dilatation balloon in an unexpanded configuration housed within a protective sheath and occupying an inner perimeter of an anatomic lumen defined by a guidewire along with a collection sheath for removing the balloon.

FIG. 4A shows the protective sheath housing the dilatation balloon traversing the length of the lesion along with the inactive collection sheath on the right side.

FIG. 4B shows the sticky dilatation balloon in its expanded configuration for trapping debris with the protective sheath having been removed and the collection sheath still inactive on the right side.

FIG. 4C shows the balloon in a partially deflated configuration after collecting debris which is shown to adhere to the outer surface and with the collection sheath activated and deployed around the sticky balloon for removing the balloon together with the debris it has collected.

FIG. 5 shows a sticky dilatation balloon in accordance with the present invention inserted through a stent traversing a lesion to provide supplementary dilatation and to clean the stent by adhering to debris in the vicinity.

FIG. 6 shows a protective sheath, housing the sticky dilatation balloon during insertion, being pulled back after implantation and placement in order to expose the sticky outer surface of the balloon and to permit inflation.

FIG. 7 shows an outer surface of a dilatation balloon in accordance with a preferred embodiment of the present invention in which the surface comprises both structurally abrasive debris-trapping elements (i.e. microhooks) and a biochemical adhesive coating, with the height of the debris-trapping elements protruding beyond the thickness of the coating.

FIG. 8 shows an alternative embodiment for a debris-trapping outer surface of the balloon in which the surface is comprised of an expandable mesh network that traps debris in between the netting.

### DETAILED DESCRIPTION OF THE INVENTION

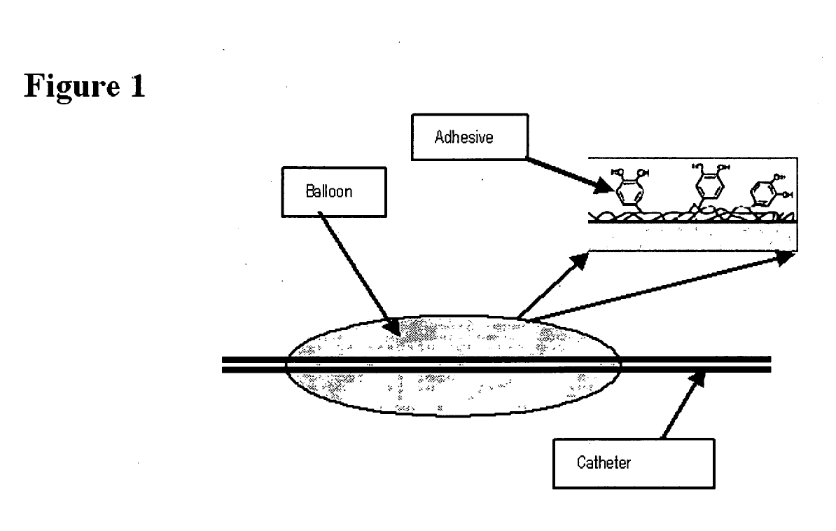

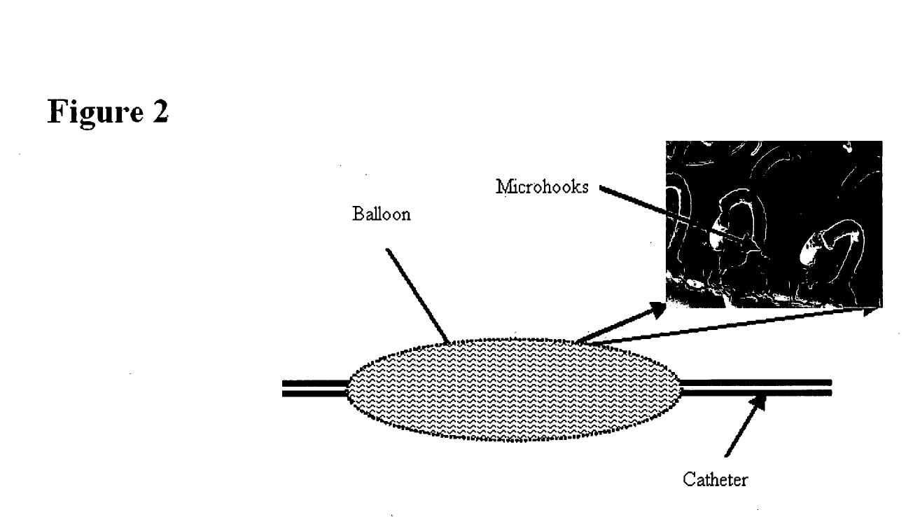

In accordance with the present invention, the preferred method for protecting against embolization in a stenting procedure is to use a sticky dilatation balloon with an outer surface that adheres to or grabs tissue debris and other extraneous materials. The balloon's outer surface can have a chemical bonded to it that adheres to unstable plaque substances such as lipids, cholesterol, thrombus, or calcium bits (see FIG. 1). Alternatively, the balloon can be covered with nano/microhooks (i.e. Velcro), nano/microfibers, hairs, split hairs, meshes, loops, foam, nano/microsuction cups, a soft or viscous gel layer, weaves, braids, aligned nano/microfibers, swirls, helical coils, nano/micro bumps, nano/micro pits, nano/micro jaws, or any other surface structure for trapping debris (see FIG. 2).

The emboli adhering/trapping surface can be part of the walls of an expandable element (i.e. inflatable balloon) or integrated onto a separate and distinct expandable cover (i.e. outer sheath) surrounding the expandable element. The cover can but need not be a flat homogenous sheet. The cover can also have a more complicated non-continuous design that “breaths” with gaps or spaces in between interconnected segments. For example the cover can be shaped like a helical coil, a series of sinusoids or zingags wires, a mesh, a braid, or an expandable tube encompassing the expandable element (inflatable balloon).

The outer cover is preferably somewhat distensible in order that it need not always be in a larger, stretched configuration necessary to accommodate the fully inflated balloon. However, the cover may be of a limited or fixed distensibility such that it only expands to a predetermined maximum diameter which acts as a check to ensure the balloon does not overinflate to cause unnecessary trauma or irritation against the luminal walls of a vessel.

Preferably, the balloon and/or its outer sheath are made of Pebax™, nylon or any other material that is both compliant and burst-resistant. Optionally, the balloon and/or sheath may be made of one or more layers with at least one layer of a non-distensible material to constrain expansion upon inflation to a fixed maximum diameter or internal pressure. Suitable non-distensible materials include polyester, polyvinylchloride PVC, polyethylene terephthalate PET, or polyethylene PE. Preferably, the outermost layer of the balloon or cover that abuts a luminal vessel wall is made of a smooth, elastomeric material, including a silicone elastomer.

When the sticky surface is provided on a separate outer cover sheath a bonding element and/or a lubricant can (but need not) be provided in between the expandable element (balloon) and the cover sheath. A bonding element or lubricant may also be provided in between the individual layers of the sheath or balloon when either one is composed of more than one layer. Lubricants reduce sliding frictions to allow the maintenance of a smooth shape. Bonding elements hold the layers together to prevent wrinkles and kinks while restraining the device to minimize the volume of space occupied. In order to provide additional protection for the inflatable element there may also be foam or cushioning incorporated within this interstitial buffer zone between the balloon and sheath. However, considering that many types of additional shock-absorbing elements will add bulk to the device and that a smaller diameter is advantageous for fitting in a lumen and crossing a lesion it is preferable to use a strong, durable, rupture-resistant material for the balloon to reduce the need for such extra padding.

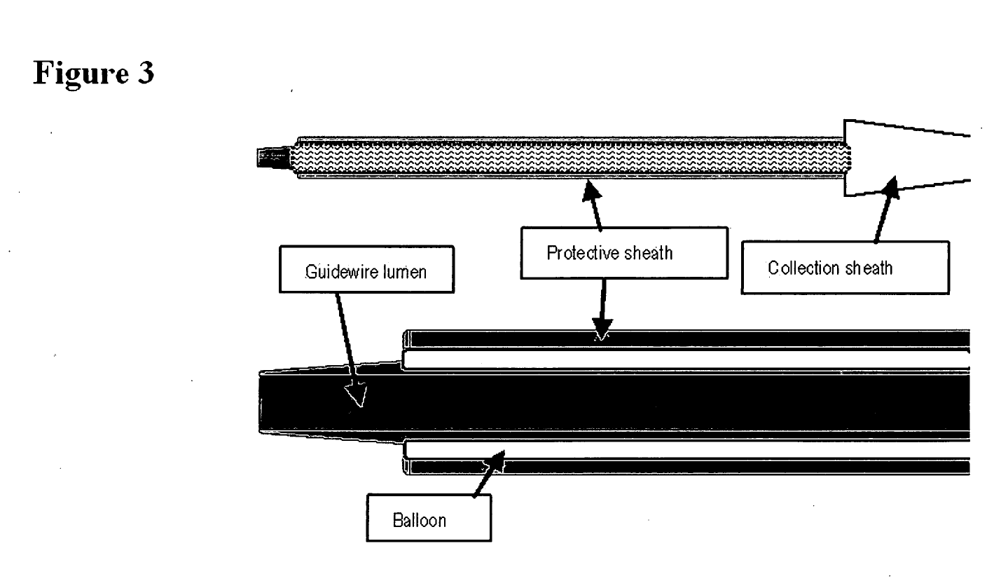

The Sticky Balloon catheter system comprises: (i) an inner tube with a guidewire lumen in the center, (ii) an annulus for inflating the balloon along the length of the catheter, (iii) a balloon near the distal end (with a sticky outer surface), (iv) optionally, a distensible cover sheath outside the balloon with a sticky outer surface, (v) a slidable, slippery protective sheath covering the collapsed balloon, and (vi) a slidable collection sheath with an expandable tip on the outside (see FIG. 3).

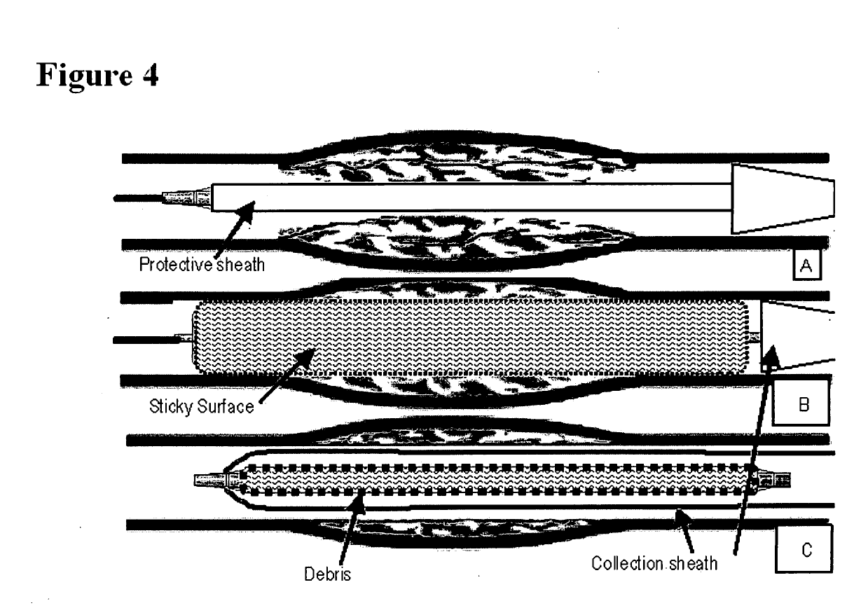

In the procedure, the Sticky Balloon catheter is advanced over a guidewire to near the proximal end margin of the target treatment site (lesion). The collection sheath tip is then expanded radially outward against the vessel to occlude blood flow proximal to the lesion. The collapsed (i.e. deflated, unexpanded) Sticky Balloon inside the protective sheath is advanced across the lesion. The protective sheath is retracted (i.e. pulled back or slid out of the way) to expose the balloon. The balloon is then inflated to dilate the lesion. Upon inflation, debris and unstable substances are easily trapped on the surface of the balloon as the pressure within the balloon places causes it to abut a lesioned vessel wall. With the debris and potential emboli trapped on its surface, the balloon is deflated and pulled back into a collection sheath that expands to accept it. Once the deflated balloon with attached debris has entered the mouth of the collection sheath, the sheath is collapsed over the balloon to retain it, sealing off the collected debris prior to longitudinal axial movement within the vessel (see FIG. 4).

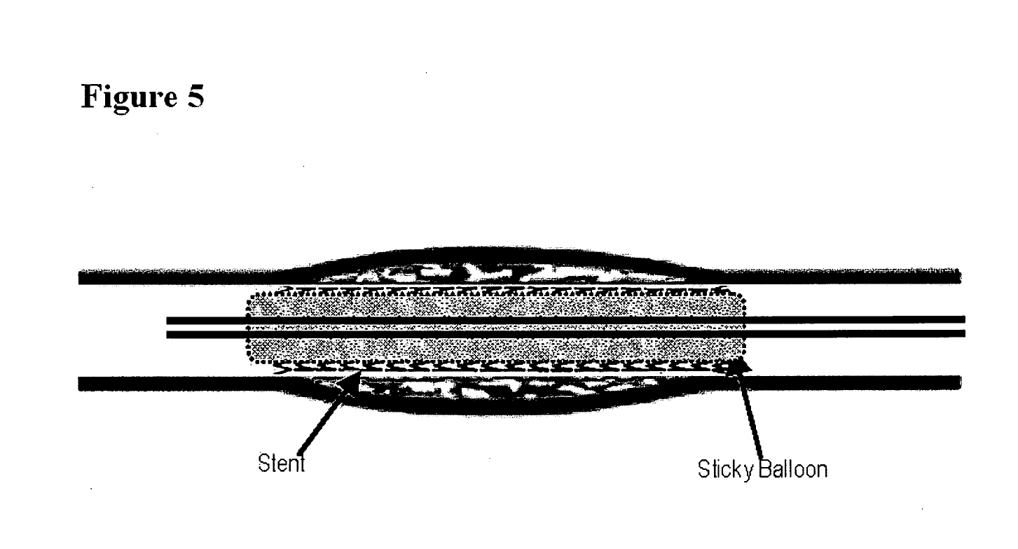

In another procedure the Sticky Balloon can be inserted within an already placed prosthetic medical device such as a stent. The radial pressure exerted outward upon inflation of the balloon can be used to expand and dilate the stent. The sticky outer surface of the balloon traps emboli as it dilates, cleaning the area around the stent (see FIG. 5). Loosened particles can become trapped in the struts of a stent. Also, porous or mesh network stents with openings therein can trap dislodged particles, at least temporarily, within these pores and openings. Using the sticky dilatation balloon of the present invention to clean the stented area intermittently significantly reduces the risk of embolization by removing particles temporarily trapped in the stent before they can be re-released into the bloodstream to cause problems in distal regions of the body. During their initial placement it is not uncommon for conventional stents to agitate the vessel wall releasing particles and getting particles trapped in their struts as the struts are stabilized within and against a vessel wall. Therefore, use of the sticky dilatation balloon in this manner as a complement to traditional stents has substantial practical application.

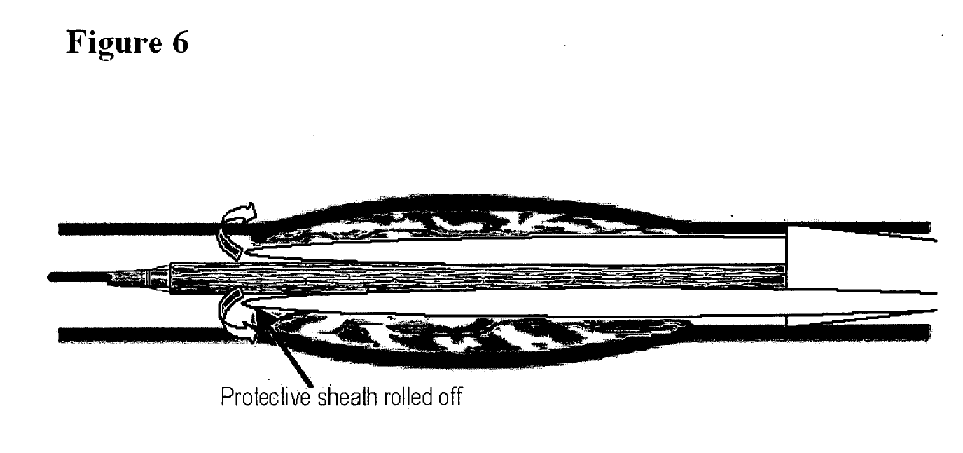

One way to facilitate the exposure of the Sticky Balloon is to have the protective sheath roll back or peel back rather than slide back (see FIG. 6). The inner walls of the protective sheath are formed of a compliant tube made of one or more material including Teflon, ePTFE, silicone, polyurethane, low density polyethylene (LDPE), nylon, isoprene, polyolefin, or any blend or copolymer thereof. The outer walls of the protective sheath have greater tensile stiffness than the inner walls. The inner walls need to be somewhat flexible to facilitate their ability to separate from the surface of the balloon and peel back. The outer walls need to be somewhat stiff to retain the balloon and to perform their function of providing a smooth, atraumatic and hemodynamic surface during balloon insertion and placement prior to inflation. The outer walls and inner walls of the protective sheath housing the balloon are connected at least at their distal ends. The outer and inner walls may additionally (but need not) be connected throughout the longitudinal lengths of their elongated bodies. The distal integration of the outer and inner walls of the protective sheath facilitates the retraction of the sheath. The outer walls are easier to physically grip and direct and may have a tab that protrudes for this purpose. The inner walls have flexibility and a lubricated non-stick surface that makes it easier for them to be detached from the sticky surface of the balloon. Using the protruding tab on the outer walls of the protective sheath to pull back the outer portion also rolls/peels back the inner wall portion to expose the sticky surface of the balloon (or alternatively, of the cover sheath outside the balloon).

The base of the sticky outer surface of the balloon or an independent cover sheath outside of it are preferably made of a hydrophobic or amphipathic material to attract the emboli which are typically hydrophobic substances including lipids, cholesterol, fibrin, fibrinogen, or thrombus. Preferred materials for the sticky surface include but not limited to polyethylene terephthalate (PET), polyethylene, polypropylene, polyesters, nylon, polyvinyl chloride (PVC), polyurethane, poly-block amide (PEBAX), isoprene, silicone, fibrin, fibrinogen, collagen or any combination thereof. For the embodiment where the sticky surface is covered with nanofibers and/or microfibers, the fibers are preferably made of PET.

Preferred materials for the adhesive chemical on the balloon surface are 3,4-L-dihydroxyphenylalanine (DOPA), albumin, collagen, fibrin, fibrinogen, cyanoacrylate, acrylated urethane, polyurethane, silicone, elastomeric rubber, epoxy, a water-activated adhesive, a pressure-activated adhesive or any combination thereof. Optionally, the propensity of the adhesive to bond to emboli can be activated by light (i.e. ultraviolet) or any other form of energy (especially electromagnetic energy) that is capable of being applied to the balloon's outer surface. The stickiness activating energy acts like a catalyst and some forms of energy may be provided from inside the balloon to radiate or diffuse through the balloon to the outer sticky surface where their impact is realized.

For applications in which the identity and composition of the emboli are known, additional biomolecules and other components can optionally be incorporated into the sticky surface in order to target specific emboli by binding to them.

Ideally, the sticky feature of the balloon is not sticky until it undergoes a certain amount of pressure such as that seen in balloon dilatation. The protective sheath can easily be pulled back to expose the balloon once the balloon is across the lesion. Then, as the balloon is inflated against the plaque, the balloon-to-plaque contact pressure activates the sticky feature. Typically at least 4 atmospheres of pressure is used to dilate lesion so the sticky feature is designed to be activated by a minimum of 3 atmospheres of pressure. In this manner the sticky feature is not activated too early such that it would interfere with positioning the balloon across the lesion or with the removal of the outer balloon sheath.

Using pressure to activate the sticky feature is best suited for mechanically abrasive sticky elements. In this manner pressure can be used to drive the mechanically abrasive elements into tissue. When the sticky feature is only or also provided by a chemical and/or biological composition coating the balloon, activation is best provided by peeling away a non-stick cover or sheath to expose the sticky inner composition to debris within the vasculature.

In another preferred embodiment, instead of (or in addition to) peeling away a cover sheath to reveal a sticky composition, the composition is shielded beneath an array of compressible or deflective elements (i.e. springs). The slippery compressible or deflective microelements (i.e. springs) should be raised above the chemical/biological composition, such as an adhesive or gel, that serves as one of the sticky features. Upon the application of a threshold amount of pressure, the raised element would deflect or compress down into the adhesive, allowing it to contact an adjacent surface such as the inner luminal wall of a vessel. Thus, the height of the compressible or deflective element should be greater than the thickness of the adhesive layer above the surface of the sticky balloon.

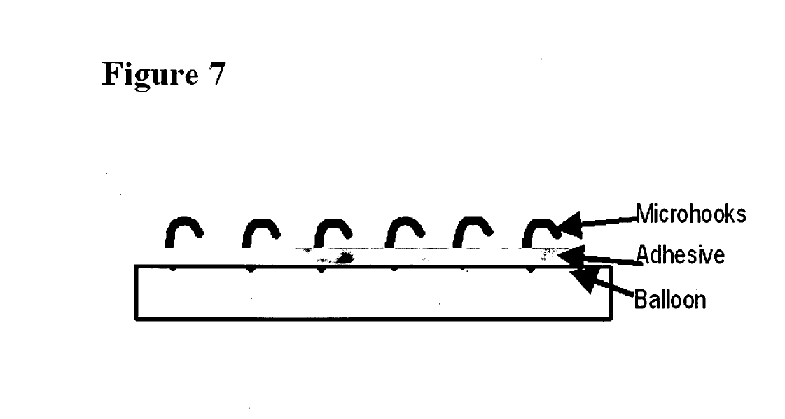

One such compressible or deflective element is a microhook that can act as a spring. Depending upon the extent of compressibility of the elements (i.e. microhooks) in relation to their height and the thickness of the adhesive layer, the pressure may cause the hooks to deflect down into the adhesive layer and/or to sink into plaque. In some embodiments, the abrasive compressible elements contact tissue first while in other embodiments the sticky adhesive composition contacts tissue first. A set of dense microhooks, like protruding Velcro hairs, can be set among a matrix of adhesive that terminates below the head of the microhooks (see FIG. 7). Both the microhooks and the biochemical adhesive matrix can act synergistically to bind a large variety of debris sizes and forms onto the balloon for removal.

The surface of the balloon can also have a thin foam layer that acts as a reservoir for the adhesive. High contact pressure compresses the foam and causes the adhesive to be exposed.

As an alternative to a balloon, an expandable sticky basket may be used. Wires made of metal or plastic can be braided together in a helical fashion such that when their lengths are condensed/shortened, they expand outward much like a balloon.

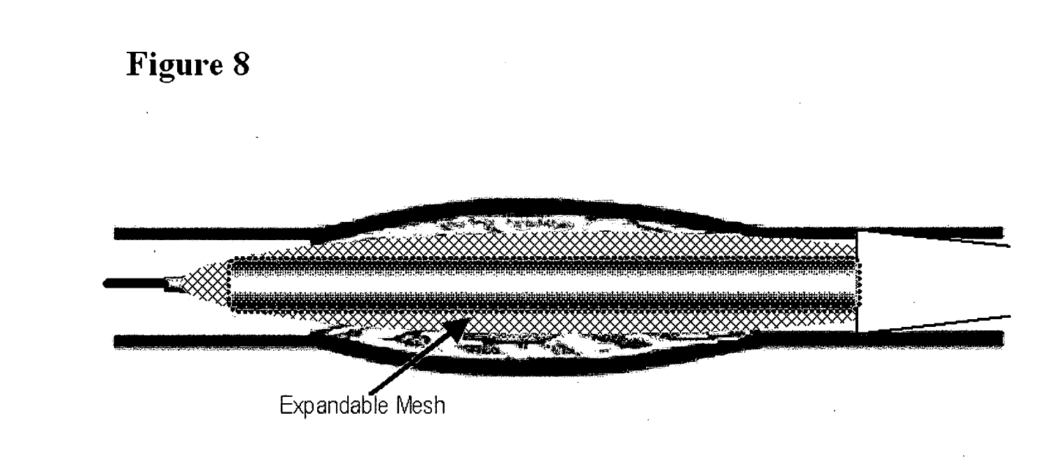

In one embodiment, the Sticky Balloon catheter has an expandable basket such as a braided mesh of ribbon wires in addition to a balloon. The basket covers the balloon (see FIG. 8). In a method of using the device, the basket is first expanded against the plaque by shortening its ends. Then, the balloon is inflated against the plaque. Once the balloon has been bonded to the unstable plaque by contact, it is then deflated. Any strings of plaque that are still stuck to the vessel wall can be cleaved and trapped by collapsing the basket by lengthening it. When the basket is collapsed, the openings in the basket become smaller and so it has the effect of scissoring and trapping the emboli.

In another embodiment, a sheath can have the sticky features on it distal to the balloon. The sheath is first delivered across the lesion. Then, the balloon is inserted inside it and inflated. This design would render a lower crossing profile for the catheter because the volume and bulkiness of the sticky features (i.e. mechanically abrasive elements and/or layered biochemical composition) is not added to the volume and bulk of the balloon during delivery. Maximum volume is reserved for when the device is appropriately positioned across a lesion site, at which point the balloon can be inflated within the sheath to drive the sticky features on the sheath into the inner luminal wall, thereby trapping debris.

In a preferred embodiment the catheter has various ports. Vacuum ports can be used to evacuate debris during the procedure. This clears filled-up areas on the surface of the balloon to recreate the sticky surface so that more debris can be trapped and collected without the need to remove the device and insert a new one. There can be ports on or near the balloon for infusing drugs or bioactive substances into the lesion. Such drugs and bioactive substances can include heparin, chlopidogrel, ticlopidine, GPVI antagonists, antagonists to the platelet adhesion receptor (i.e. GP1b-V-IX), antagonists to the platelet aggregation receptor (i.e. GPIIb-IIIa), enoxaparin, dalteparin, hirudin, bivalirudin, argatroban, danparoid, Tissue Factor Pathway Inhibitor (TFPI), vascular endothelial growth factor (VEGF), angiopoietin-1, fibroblast growth factor (bFGF), antisense dexamethasone, angiopeptin, acetylsalicylic acid, nicotine, steroids, ibuprofen, antimicrobials or antibiotics (i.e. Actinomycin D), tissue plasma activators, antifibrosis agents, high density lipoprotein, estradiol, agents that affect vascular smooth muscle cell (VSMC) proliferation or migration (i.e. transcription factor E2F1), Batimistat, Biolimus, Everolimus, Halofuginon, Tacrolimus, Taxan™, Taxol™, Tranilast, Translast, Rapamycin, rapamycin analogs, and Zotarolimus.

The balloon can have reservoirs for these drugs that allow them to ooze out into the lesion upon application of a certain threshold contact pressure. A preferred pressure threshold for releasing the drug is 6 atmospheres, the minimum pressure used in angioplasty and stent dilatation.

The balloon can also be covered with scales or other slippery microstructures that open up to expose the sticky structures/adhesive upon balloon inflation. This way a protective sheath is not required resulting in lower crossing profile.

In a preferred embodiment, the sticky surface on the balloon described herein can be peeled off of the balloon and left behind as a coating on the lesion to stabilize the unstable plaque and encourage endothelization. When used as a transferable coating, the sticky surface preferably forms a coating of aligned nanofibers. The inner fibers do not have the sticky features described herein but the outer fibers do so that they can adhere to the lumen wall. The inner fibers preferably have smooth surfaces so as not to interfere with healthy blood flow. Preferably, the outer fibers are aligned approximately perpendicular to the axis of the vessel and the inner fibers are aligned approximately parallel to the axis of the vessel. This type of alignment pattern permits the outer fibers to adhere more securely to the inner luminal wall of a vessel and permits the inner fibers to facilitate normal hemodynamics and to encourage endothelization.

The nanofibers are preferably made of bioresorbable or biodegradable materials such as magnesium alloys, hydroxyapatite, and polymers such as polylactic acid (PLA) compounds, polyglycolic (PGA) compounds, polycaprolactone, polyvalerate, polyhydroxybutyriate (PHB), polydioxanone, polyanhydrides, poly-ortho esters, polyiminocarbonates, polyetheresters, silk, modified collagen, any blend of the aforementioned polymers, and any co-polymers of the aforementioned polymers (i.e. PGA-PLA).

The aligned nanofiber coating for encouraging endothelization can itself be coated or impregnated with drugs or substances to minimize thrombus formation. Preferred candidates for drugs that may be incorporated therein include: heparin, chlopidogrel, ticlopidine, GPVI antagonists, antagonists to the platelet adhesion receptor (i.e. GP1b-V-IX), antagonists to the platelet aggregation receptor (i.e. GPIIb-IIIa), enoxaparin, dalteparin, hirudin, bivalirudin, argatroban, danparoid, or Tissue Factor Pathway Inhibitor (TFPI). The deposited aligned nanofiber coating can also be coated or impregnated with pro-endothelization substances including vascular endothelial growth factor (VEGF), angiopoietin-1, and phosphorylcholine. Any combination of the drugs and other substances recited above with or without the incorporation of other materials can be used to minimize thrombus formation.

The deposited aligned nanofiber coating can also be coated or embedded with immunological suppressants or anti-proliferative drugs (instead of or in addition to anticoagulants and thrombus inhibitors) including Taxol™, Everolimus, and Rapamycin to minimize restenosis. Other substances for this purpose can include: Biolimus, Zotarolimus, Tacrolimus, fibroblast growth factor (bFGF), rapamycin analogs, antisense dexamethasone, angiopeptin, Batimistat, Tranilast, Halofuginon, acetylsalicylic acid, hirudin, steroids, ibuprofen, antimicrobials or antibiotics (i.e. Actinomycin D), tissue plasma activators, and agents that affect VSMC proliferation or migration (i.e. transcription factor E2F1).

An alternative method for protecting against embolization in a stenting procedure is to use a dilatation balloon (Sticky Balloon) with an outer surface that adheres to tissue debris. The balloon's outer surface can have tissue adhesive bonded to it or it can be covered with microhooks like Velcro for trapping tissue debris. In a procedure, the Sticky Balloon is collapsed into a smooth restraining sheath and advanced across the lesion. The restraining sheath is then pulled back to expose the balloon. The balloon is inflated to dilate the lesion. Tissue debris or emboli are trapped on the surface of the balloon. The balloon is deflated and pulled back into an expandable tube connected to the collection sheath. The Sticky Balloon can also be used inside a stent to expand the stent and trap emboli as it dilates. The microhooks can also be disposed on an expandable sheath over the dilatation balloon instead of it being directly on the balloon itself. Preferably, when the adhesive or mechanical abrasive element (i.e. microhooks) are provided on a separate sheath it should be securely bonded to the balloon such that the expansion and contraction of the balloon induces corresponding movement in the sheath. A secure balloon-adhesive sheath interface also prevents air bubbles and kinks from interfering with the dilatation and cleaning processes.

A dumbbell shaped balloon is preferred in order to better trap the debris. The dumbbell shaped balloon improves debris retention by preventing it from escaping at sites immediately proximal and distal to the lesion. With a dumbbell shaped balloon, even if the debris does not immediately adhere to the adhesive surface it is at least prevented from being re-distributed through the body. The oblong ends of a dumbbell shaped balloon function to prevent debris and particulates from leaving the vicinity of the sticky balloon, thereby permitting the sticky surface with additional chances to grab onto them. If the sticky surface is too full with other debris already that it cannot adhere to any more debris then either it should be removed and a new sticky balloon inserted or preferably, vacuum suction should be activated to clean off the surface of the positioned balloon. Vacuum suction is preferable when there is excess debris that cannot be trapped on the balloon's surface because it is already too crowded. Suction is preferable because activating it does not disrupt the placement of the bulging ends of a dumbbell shaped balloon. Removing the dumbbell shaped balloon may permit excess debris to escape from the vicinity of the lesion before a new balloon with available adhesive surface can be inserted.

Preferably, the balloon catheter has its own occlusion balloon(s) on it to occlude blood flow proximal and/or distal to the lesion prior to introduction of the sticky balloon across the lesion through the catheter.

What is claimed is:

1. A device for dilating narrowed blood vessels comprising:
a proximal end and a distal end;
a balloon on a catheter; and
an adhesive surface outside of the balloon.

2. The device of claim 1, further comprising at least one port within the catheter for introducing or removing a gas, a liquid, a solid or a hybrid substance.

3. The device of claim 2, wherein at least one port is for providing vacuum suction to remove debris trapped to the adhesive surface in order to create space for more debris to be trapped.

4. The device of claim 2, wherein at least one port is for locally delivering a therapeutic agent or drug to a narrowed region of a blood vessel.

5. The device of claim 1, wherein the balloon is dumbbell shaped.

6. The device of claim 1, further comprising shielding scales or protective microstructures outside the adhesive surface outside of the balloon;
wherein the scales or microstructures are configured to shield or protect the adhesive surface during delivery of the device such that during delivery the non-exposed adhesive surface is prevented from collecting debris and filling up too quickly; and
the scales or microstructures are also configured to peel back, retract, and/or dissolve after implantation of the device in a desired target location in order to expose the adhesive surface so that it then collects material.

7. The device of claim 6, wherein the scales or microstructures are configured to peel back, retract, and/or dissolve upon inflation of the balloon.

8. The device of claim 1, further comprising a treatment element extendable from the catheter in a vicinity of the balloon for providing therapy to and severing tissue.

9. The device of claim 8, wherein the treatment element is selected from the group consisting of: a blade, an electrode, and a radiation-emitting probe.

10. The device of claim 1, wherein the adhesive surface exists on the balloon itself.

11. The device of claim 1, further comprising an expandable sheath covering the balloon, wherein the adhesive surface exists on the expandable sheath.

12. The device of claim 1, wherein the balloon is both collapsable and inflatable and the adhesive surface is configured to adhere or stick to tissue and debris; and
further comprising a retractable sheath covering the balloon in its collapsed configuration during delivery.

13. The device of claim 1, wherein the adhesive surface comprises a sticky chemical and/or biological composition.

14. The device of claim 13, further comprising a thin layer of non-stick, porous, compressible foam over the sticky composition, wherein upon application of a threshold amount of pressure, the sticky composition is dispensed through the foam layer to bond tissue fragments and debris to the balloon.

15. The device of claim 1, wherein the adhesive surface comprises mechanically abrasive elements.

16. The device of claim 13, wherein the adhesive surface further comprises mechanically abrasive elements in addition to the sticky chemical and/or biological composition.

17. The device of claim 16, wherein the mechanically abrasive elements protrude from the adhesive surface and the sticky chemical and/or biological composition is coated on the protruding mechanically abrasive elements.

18. The device of claim 16, wherein the mechanically abrasive elements protrude from the surface of the sticky composition and are compressible like springs, such that upon application of a threshold amount of pressure they deflect into the composition to expose it for binding debris.

19. The device of claim 13, wherein the sticky composition is bonded to a surface beneath it.

20. The device of claim 19, wherein the bonding is selected from the group consisting of: covalent bonding, hydrogen bonding, and a combination thereof.

21. The device of claim 13, wherein the sticky composition takes the form of 3,4-L-dihydroxyphenylalanine.

22. The device of claim 13, wherein the sticky composition takes the form of poly(dopamine methacrylamide-co-methoxyethylacrylate).

23. The device of claim 13, wherein the sticky composition comprises at least one element selected from the group consisting of: dihydroxyphenyl, hydroxyphenylalanine, albumin, collagen, fibrin, fibrinogen, cyanoacrylate, acrylated urethane, polyurethane, silicone, elastomeric rubber, epoxy, a water-activated adhesive, and a pressure-activated adhesive.

24. The device of claim 15, wherein the mechanically abrasive elements take the form of microhooks.

25. The device of claim 12, wherein the retractable sheath is configured to be rolled back to expose the adhesive surface.

26. The device of claim 1, further comprising an expandable element on the catheter for occluding blood flow beyond the balloon.

27. The device of claim 1, further comprising an expandable filter on the catheter distal to the balloon for filtering material received by the balloon.

28. The device of claim 1, wherein the adhesive surface is bound to a coating that detaches from the balloon and transfers to bind to a luminal wall of a vessel following inflation and deflation of the balloon.

29. The device of claim 28, wherein the detachable coating comprises aligned microfibers or aligned nanofibers.

30. The device of claim 29, wherein the aligned fibers are arranged such that outer fibers are approximately perpendicular to an axis of the vessel and inner fibers are approximately parallel with an axis of the vessel.

31. The device of claim 29, wherein the aligned fibers are biodegradable.

32. The device of claim 29, wherein the aligned fibers are composed of a material selected from the group consisting of: polylactic acid, polyglycolic acid, polycaprolactone, polyvalerate, polyhydroxybutyriate, polydioxanone, polyanhydrides, poly-ortho esters, polyiminocarbonates, polyetheresters, silk, modified collagen, any blend of the aforementioned polymers, and any co-polymers of the aforementioned polymers.

33. The device of claim 28, further comprising a bioactive substance embedded within the detachable coating.

34. The device of claim 33, wherein the bioactive substance comprises at least one element selected from the group consisting of: heparin, ticlopidine, chlopidogrel, enoxaparin, dalteparin, hirudin, bivalirudin, argatroban, danparoid, Tissue Factor Pathway Inhibitor (TFPI), rapamycin analogs, antisense dexamethasone, angiopeptin, nicotine, acetylsalicylic acid, steroids, ibuprofen, antimicrobials, antibiotics, tissue plasma activators, antifibrosis agents, high density lipoprotein, and estradiol.

35. The device of claim 33, wherein the bioactive substance comprises at least one element selected from the group consisting of: Batimistat, Biolimus, Everolimus, Halofuginon Rapamycin, Tacrolimus, Taxan™, Taxol™, Tranilast, Translast, and Zotarolimus.

36. A device for dilating narrowed blood vessels comprising:
a balloon on a catheter; and
at least one debris trapping structure outside of the balloon.

37. The device of claim 36, wherein the debris trapping structure exists on an outside surface of the balloon itself.

38. The device of claim 36, further comprising an expandable sheath covering the balloon, wherein the debris trapping structure exists on the expandable sheath.

39. The device of claim 36, wherein the balloon is both collapsable and inflatable and the debris trapping structure is configured to adhere or stick to tissue and debris; and
further comprising a retractable sheath covering the balloon in its collapsed configuration during delivery.

40. The device of claim 36, wherein the debris trapping structure is a microhook.

41. The device of claim 40, wherein the microhook is 12 microns to 250 microns in thickness and 1000 microns to 4000 microns in height.

42. The device of claim 36, wherein the debris trapping structure comprises at least one element selected from the group consisting of: hairs, loops, nano/micro fibers, aligned nano/micro fibers, meshes, nano/micro suction cups, foam, soft gel layer(s), viscous gel layer(s), weaves, braids, swirls, helical coils, nano/micro bumps, nano/micro pits, nano/micro jaws, and nano/micro tubes.

43. The device of claim 36, wherein the debris trapping structure is hydrophobic.

44. The device of claim 36, wherein the debris trapping structure is amphipathic.

45. The device of claim 36, wherein the debris trapping structure is a hair with split ends.

46. The device of claim 36, wherein the debris trapping structure is coated with an adhesive layer.

47. The device of claim 46, wherein a thickness of the adhesive layer is less than a height of the debris trapping structure(s).

48. The device of claim 46, wherein the adhesive layer comprises 3,4-L-dihydroxyphenylalanine.

49. The device of claim 46, wherein the adhesive layer comprises poly(dopamine methacrylamide-co-methoxyethylacrylate).

50. The device of claim 46, wherein the adhesive layer comprises at least one element selected from the group consisting of: dihydroxyphenyl, hydroxyphenylalanine, albumin, collagen, fibrin, fibrinogen, cyanoacrylate, acrylated urethane, polyurethane, silicone, elastomeric rubber, epoxy, a water-activated adhesive, and a pressure-activated adhesive.

51. The device of claim 36, wherein the debris trapping structure is an expandable mesh.

52. The device of claim 39, wherein the retractable sheath is configured to be rolled back to expose the debris trapping structure.

53. The device of claim 36, further comprising an expandable element on the catheter for occluding blood flow beyond the balloon.

54. The device of claim 36, further comprising an expandable filter on the catheter distal to the balloon for filtering material received by the balloon.

55. The device of claim 36, wherein the debris trapping structure is bound to a coating that detaches from the balloon and transfers to bind to a luminal wall of a vessel following inflation and deflation of the balloon.

56. The device of claim 55, wherein the detachable coating comprises aligned microfibers or aligned nanofibers.

57. The device of claim 56, wherein the aligned fibers are arranged such that outer fibers are approximately perpendicular to an axis of the vessel and inner fibers are approximately parallel with an axis of the vessel.

58. The device of claim 56, wherein the aligned fibers are biodegradable.

59. The device of claim 56, wherein the aligned fibers are composed of a material selected from the group consisting of: polylactic acid, polyglycolic acid, polycaprolactone, polyvalerate, polyhydroxybutyriate, polydioxanone, polyanhydrides, poly-ortho esters, polyiminocarbonates, polyetheresters, silk, modified collagen, any blend of the aforementioned polymers, and any co-polymers of the aforementioned polymers.

60. The device of claim 55, further comprising a bioactive substance embedded within the detachable coating.

61. The device of claim 60, wherein the bioactive substance comprises at least one element selected from the group consisting of: heparin, ticlopidine, chlopidogrel, enoxaparin, dalteparin, hirudin, bivalirudin, argatroban, danparoid, Tissue Factor Pathway Inhibitor (TFPI), rapamycin analogs, antisense dexamethasone, angiopeptin, nicotine, acetylsalicylic acid, steroids, ibuprofen, antimicrobials, antibiotics, tissue plasma activators, antifibrosis agents, high density lipoprotein, and estradiol.

62. The device of claim 60, wherein the bioactive substance comprises at least one element selected from the group consisting of: Batimistat, Biolimus, Everolimus, Halofuginon, Rapamycin, Tacrolimus, Taxan™, Taxol™, Tranilast, Translast, and Zotarolimus.

63. A device for dilating narrowed blood vessels comprising a basket on a catheter,
wherein the basket is composed of wires and the basket is configured to be reversibly shortened and lengthened by manipulating the wires,
wherein shortening the basket expands a center part of the basket to dilate a lumen and opens spaces between wires to receive tissue and debris, and
wherein lengthening the basket brings the wires closer together to reduce the spaces between them and causes a scissoring effect between wires that severs tissue.

64. A method for dilating narrowed blood vessels using the device according to claim 36, comprising the steps of:
placing a balloon covered with at least one debris trapping structure within a narrow region defined by an inner lumen of a blood vessel;
inflating the balloon; and
deflating the balloon.

65. The method of claim 64, further comprising the step of collapsing a cover over the balloon after deflating the balloon.

66. The method of claim 64, wherein the debris trapping structure is a sticky coating.

67. The method of claim 66, wherein during the step of deflating the balloon the coating detaches from the balloon and transfer to bind to a luminal wall of the vessel.

68. The method of claim 64, further comprising the step of pulling back a sheath covering the balloon in its collapsed condition in order to uncover the balloon before inflating.

69. The method of claim 64, further comprising the step of rolling off a sheath covering the balloon in its collapsed condition in order to uncover the balloon before inflating.

70. The method of claim 64, wherein the debris trapping structure is a mesh, and further comprising the steps of expanding the mesh at the same time as or prior to expansion of the balloon and collapsing the mesh at the same time as or after deflating the balloon.

71. The method of claim 64, further comprising the steps of extending and expanding an element to occlude blood flow beyond the balloon prior to inflating the balloon to prevent blood flow from interfering with inflation.

72. The method of claim 64, further comprising the step of deploying an expandable filter distal to the balloon prior to inflating the balloon to reduce an initial flux of materials directed at the balloon and to prevent the debris trapping structure(s) from becoming filled too quickly.

73. A method for dilating narrowed blood vessels using the device according to claim 61, comprising the steps of:
inserting a catheter with a wire basket on its distal end into a narrowed blood vessel;
shortening a longitudinal length of the wire basket to expand spaces between adjacent wires thereby radially expanding a central region of the basket;
trapping excess tissue and other debris within the expanded spaces between adjacent wires;
elongating the longitudinal length of the wire basket to contract spaces between adjacent wires thereby radially shrinking the central region of the basket;
severing trapped excess tissue and other debris as adjacent wires move together such that the tissue and debris falls within the basket; and
removing the severed tissue and debris from the basket.
